(19) United States
(12) Patent Application Publication
(10) Pub. No.: US 2024/0052249 A1
(43) Pub. Date: Feb. 15, 2024

(54) PROCESS

(71) Applicant: UNIVERSITETET I OSLO, Oslo (NO)

(72) Inventors: Unni OLSBYE, Oslo (NO); Jingxiu XIE, LA Groningen (NL)

(21) Appl. No.: 18/265,829

(22) PCT Filed: Dec. 17, 2020
(86) PCT No.: PCT/EP2021/086514

§ 371 Date: Jun. 7, 2023

(30) Foreign Application Priority Data
Dec. 17, 2020 (GB) 2020054.9

(51) Int. Cl. C10G 49/08

(52) U.S. Cl. CPC C10G 49/08; C10G 2300/4012; C10G 2300/4006; C10G 2400/20

(57) ABSTRACT

The invention provides a process for preparing olefins from a mixed gaseous feed stream, wherein said mixed gaseous feed stream comprises three or more components selected from the group consisting of carbon dioxide, carbon monoxide, hydrogen, methanol and dimethyl ether, said process comprising contacting the mixed gaseous feed stream with a catalyst of formula (I): M(II)_{χ}Al1_{1-χ}PO4 (I), wherein M(II) is a divalent metal ion; and x=0.002 to 0.5

This invention concerns a process for preparing olefins. In particular, it relates to a process for preparing olefins from a mixed gaseous feed stream comprising three or more components selected from the group consisting of carbon dioxide, carbon monoxide, hydrogen, methanol and dimethyl ether, said process comprising contacting the mixed gaseous feed stream with a divalent metal based catalyst. The divalent metal based catalyst is a particular class of metal heteroatom-containing aluminophosphate (MAPO) molecular sieve. The invention also relates to the use of the MAPO molecular sieves as a catalyst in a process for preparing olefins.

### BACKGROUND

The conversion of methanol to olefins (MTO) using zeolite/zeotype catalysts provides a viable solution to the production of chemicals from alternative carbon raw materials, including natural gas, CO₂, biomass and municipal waste. The industrial process typically operates at 350-500° C. and 1 bar, using silicoaluminophosphate (SAPO-34) and zeolite (ZSM-5) catalysts. High selectivity towards C₂-C₄ olefins is achieved with SAPO-34 is due to its topology, which limits product effusion to molecules smaller than 3.8 Å. On the other hand, high selectivity towards propylene is attained with ZSM-5 by recycling of products and operation at higher temperatures (>500° C.) to facilitate cracking of the hydrocarbon products.

On the molecular scale, MTO product distribution is governed by a delicate balance between relative diffusivities, competitive adsorption and reaction on the internal surface of the microporous catalysts. The reaction path is dominated by a dual-cycle mechanism, in which alkenes and arenes (the hydrocarbon (HC) pool species) are methylated, and subsequently cracked or dealkylated to form light olefins.

In addition to the zeolite/zeotype topology, the number/strength/distribution of acidic sites, lattice defects and crystal size/morphology influence MTO catalyst performance. Reaction conditions such as temperature, methanol partial pressure and contact time, are also paramount to optimal catalyst performance.

Owing to its industrial success at lower temperatures, SAPO-34 has been the obvious choice as part of a bifunctional catalyst for the direct conversion of CO/CO₂/H₂ feeds to lower olefins via oxygenate intermediates under tandem reaction conditions. For thermodynamics to favour the CO/CO₂ conversion to methanol (MeOH), this reaction should operate at low temperatures (<300° C.) and high pressures (>20 bar). However, the MTO catalysts discussed above were found not to be effective at such low temperatures because the dealkylation of aromatics is unfavourable resulting in fast deactivation. There remains a need, therefore, to provide new catalysts for use in processes for the production of olefins directly from mixed gaseous feed streams. Such mixed gaseous feed streams could originate from e.g. product stream recycling after separation of hydrocarbon products, which is common industrial practice. It could also originate from a configuration of reactors and/or reaction zones in series, where part of the CO/CO₂/H₂ feed is preconverted into methanol, dimethyl ether or a mixture of both in a first reactor or reaction zone. All or part of the effluent gas stream containing CO, CO₂, H₂, MeOH, DME is then fed over an olefin-producing catalyst in the second reactor or reaction zone, which concerns the invention here.

The present inventors have surprisingly found that a particular class of metal heteroatom-containing aluminophosphate (MAPO) molecular sieves containing a divalent heteroatom (M) offer an attractive solution. In particular, the MAPO materials offer improved conversion to olefins under equivalent mixed feed reactor conditions compared to the conventionally used materials comprising tetravalent heteroatoms (such as SAPO). Furthermore, improved selectively for olefins over undesirable side products, such as paraffins, is observed.

### SUMMARY OF INVENTION

Thus, viewed from a first aspect the invention provides a process for preparing olefins from a mixed gaseous feed stream, wherein said mixed gaseous feed stream comprises three or more components selected from the group consisting of carbon dioxide, carbon monoxide, hydrogen, methanol and dimethyl ether, said process comprising contacting the mixed gaseous feed stream with a catalyst of formula (I):

M(II)ₓAl₁₋ₓPO₄ (I)
wherein M(II) is a divalent metal ion; and
x=0.002 to 0.5.

Viewing from a further aspect, the invention provides the use of a compound of Formula (I) as defined herein as a catalyst in a process for preparing olefins from a mixed gaseous feed stream as defined herein.

### Definitions

The term “molecular sieve” used herein refers to a class of crystalline materials with defined arrangements of cavities, channels and/or pores. The molecular sieve framework or topology is characteristic of the specific type of molecular sieve. A framework type or topological type is unique and is provided with a unique three letter code by the IZA (International Zeolite Association). Framework types or topological types are not only defined by composition, but also by the arrangement of the atoms that bound the cavities, channels and/or pores that make up the structure. Molecular sieves can usually be identified by their x-ray diffraction (XRD) pattern.

### DETAILED DESCRIPTION OF INVENTION

The present invention relates to a process for preparing olefins from a mixed gaseous feed stream comprising three or more components selected from the group consisting of carbon dioxide, carbon monoxide, hydrogen, methanol and dimethyl ether, said process comprising contacting the mixed gaseous feed stream with a catalyst, wherein the catalyst is a particular class of metal heteroatom-containing aluminophosphate (MAPO) molecular sieve.

### Metal Heteroatom-Containing Aluminophosphate (MAPO)

The MAPO employed as a catalyst in the processes of the invention has the formula (I):

M(II)ₓAl₁₋ₓPO₄ (I)
wherein M(II) is a divalent metal ion; and
x=0.002 to 0.5.

M(II) may be any divalent metal ion (i.e. a metal ion in the +2 oxidation state) and is preferably selected from the group consisting of Be, Mg, Ca, Zn, Mn, Fe, Co, Ni, Cu, Cd and Sr. More preferably, M(II) is Mg, Co, Zn, Fe, Ni, Cu or Mn, even more preferably Mg, Co or Zn, especially Mg.

In Formula (I) above, x is preferably 0.005 to 0.2, such as 0.01 to 0.18.

The lower positive charge of M(II) compared to Al(III) is compensated by a cation, most commonly a proton (H+) ion-exchanged onto the oxygen that bridges the M(II)-O—P pair, thereby creating a Brønsted acid site.

The MAPO is a microcrystalline molecular sieve comprising a pore structure. The pore structure will be familiar to those skilled in the field. The MAPOs employed in the invention preferably have an 8-member ring pore opening.

The topology of the MAPO is not limited and may, for example, be selected from AEI, CHA, AFN and SAV. Such topologies are well known in the art and are defined by the Structure Commission of the International Zeolite Association (IZA). Preferably, the MAPO has AEI topology.

As used herein, the term “AEI” refers to an AEI topological type as recognized by the International Zeolite Association (IZA) Structure Commission and means a MAPO in which the primary crystalline phase is AEI. Other crystalline phases may also be present. In particular, those who are skilled in the field will recognize that MAPOs consisting mainly of AEI or CHA phases will often contain stacking faults or intergrowth with the other phase, and that it is difficult to assign a specific percentage of the material to one or the other structure. By “primary crystalline phase” with respect to AEI, it is meant that the Powder X-Ray Diffraction (PXRD) pattern of the material after calcination is dominated by peaks assigned to the AEI crystal structure.

More particularly, the MAPO is an isostructural member of the AEI grouping known as MAPO-18.

The M(II) content of the MAPO is typically at least 0.01 wt %, preferably at least 0.1 wt %, such as at least 0.5 wt %, based on the total weight of the MAPO.

Whilst it is within the ambit of the invention for more than one M(II) to be present in the MAPO it is preferred if a single M(II) is used.

Other than the M(II) and the aluminium, the MAPO used in the present invention preferably does not include any other metal atoms, i.e., heteroatoms, in significant amounts, although trace amounts of other metals may result from the preparation process. As the term is used herein, “trace amounts” represent amounts less than 1.0 wt %, preferably less than 0.5 wt %, more preferably less than 0.1 wt %, and most preferably less than 0.005 wt %, relative to the total weight of the MAPO.

In all embodiments of the invention, it is preferred if the MAPO does not comprise silicon.

The MAPO may be prepared by any suitable method known in the art.

Example methods include hydrothermal synthesis, solvothermal synthesis or methods involving post-synthesis modification via e.g. steaming, acid leaching, impregnation, ion migration and/or ion exchange.

The reaction mixture for the MAPO hydrothermal synthesis process typically contains at least one source of phosphorous, at least one source of alumina, at least one source of the divalent metal M(II) and at least one structural directing agent useful in forming the MAPO.

A number of aluminum compounds and their mixtures are suitable for use as the aluminum component in the present invention. A source of alumina can comprise for example an aluminum alkoxide, such as aluminum isopropoxide, aluminum tri-ethoxide, aluminum tri-n-butoxide and aluminum tri-isobutoxide, an aluminum oxide, an aluminum phosphate, aluminum hydroxide, sodium aluminate, (pseudo)boehmite, hydrated alumina, organoalumina, aluminum hydroxy chloride, colloidal alumina, and mixtures thereof. Preferably, the aluminum component comprises a material selected from the group consisting of aluminum hydroxide, boehmite and pseudoboehmite, most preferably boehmite (alumina hydrate).

A source of phosphorus can comprise, but is not limited to, orthophosphoric acid, phosphorus acid, phosphoric acid, organic phosphate such as triethyl phosphate and trimethylphosphate, aluminophosphate, and mixtures thereof. Preferred is a source of phosphorus comprising a material selected from the group consisting of phosphoric acid (such as the commercially available 85 wt % phosphoric acid in water), and orthophosphoric acid. Alternatively, phosphorus oxides (P2O3, P2O4, P2O5 and POCI3) may be used, preferably after they are dissolved in a suitable solvent such as water.

A source of the divalent M(II) may be any precursor thereof, such as for example nitrates, acetates, oxalates, sulphates, organometallic complexes and combinations thereof. Preferably the source is an acetate, more preferably a M(II) acetate tetrahydrate.

The structural directing agent is any compound which provides the desired template for MAPO formation, preferably selected from N,N-dimethyl-3,5-dimethyl (DMDMP), N,N-diethyl-2,6-dimethylpiperidine (DEDMP), N,N-dimethyl-2,6-dimethylpiperidine, N-ethyl-N-methyl-2,6-dimethylpiperidine, N,N-diisopropylethylamine, tetraethylammonium hydroxide, triethylamine and combinations thereof, and any variation thereof. Further examples include cyclic quaternary ammonium compounds or any other organic molecule, such as for example, any amine, quaternary ammonium, phosphine, phosphonium, phosphazene or combinations thereof. Preferably, the structural directing agent is N,N-diisopropylethylamine.

Generally, the reaction mixture is maintained at an elevated temperature until the MAPO crystals are formed. The hydrothermal crystallization is usually conducted under autogenous pressure, at a temperature between about 75-220° C., for example between about 180 and 210° C., for duration of several hours, for example, about 0.1-20 days, and preferably from about 0.25-10 days. Preferably, the MAPO is prepared using stirring or agitation.

During the hydrothermal crystallization step, crystals of the MAPO can be allowed to nucleate spontaneously from the reaction mixture. Once the crystals have formed, the solid product is typically separated from the reaction mixture by standard separation techniques such as filtration or centrifugation. The crystals are normally water-washed and then dried, for several second to a few minutes (e.g., 5 second to 10 minutes for flash drying) or several hours (e.g., about 4-24 hours for oven drying at 75-150° C.), to obtain as-synthesized MAPO crystals. The drying step can be performed at atmospheric pressure or under vacuum.

The MAPO crystals produced in accordance with the methods described herein can have a mean crystalline size of 0.05 to 5 μm, for example about 0.1 to about 4 μm, such as 0.2 to about 3 μm.

Typically, the MAPO is then calcined. The term “calcine”, or “calcination”, means heating the material in air or oxygen. The temperatures used in calcination depend upon the components in the material to be calcined and generally are between about 400° C. to about 900° C. for approximately 1 to 8 hours. In some cases, calcination can be performed up to a temperature of about 1200° C. In applications involving the processes described herein, calcinations are generally performed at temperatures from about 400° C. to about 700° C. for approximately 1 to 8 hours, preferably at temperatures from about 400° C. to about 650° C. for approximately 1 to 4 hours.

### Process

The process of the invention may be any suitable process known in the art for the production of olefins from a mixed gaseous feed stream comprising three or more components selected from the group consisting of carbon dioxide, carbon monoxide, hydrogen, methanol and dimethyl ether. The skilled person will be familiar with such processes and the instruments employed therein.

The mixed gaseous feed stream used in the process of the invention may be any gas stream comprising three or more components selected from the group consisting of carbon dioxide, carbon monoxide, hydrogen, methanol and dimethyl ether. Optionally one or more further gases may be present.

It is particularly preferred if one of the components of the mixed gaseous feed stream is methanol or dimethyl ether.

In a preferred embodiment, the feed stream comprises hydrogen (H₂) and CO₂. It is particularly common for the gaseous feed stream to further comprise carbon dioxide (CO).

In an alternative embodiment, the feed stream comprises hydrogen (H₂), CO and methanol (MeOH).

In one embodiment, the feed stream may comprise H₂, CO, MeOH and dimethyl ether. In another embodiment, the feed stream may comprise H₂, CO, methanol (MeOH) and CO₂. In yet another embodiment, the feed stream may comprise H₂, CO, methanol (MeOH), dimethyl ether (DME) and CO₂.

In embodiments wherein the feed stream comprises H₂ and CO₂ (and optionally CO), the ratio of H₂:CO₂ content in vol % is typically in the range 0.5:1 to 20:1, such as 2:1 to 10:1, wherein the vol % is relative to the total amount of gas present.

The feed stream may comprise 0.8 to 11 vol % MeOH, preferably 2 to 8 vol % MeOH, relative to the total amount of gas present.

The feed stream may comprise 0.02 to 36 vol % of dimethyl ether, preferably 0.02 to 5 vol % dimethyl ether, relative to the total amount of gas present.

The feed stream may comprise 5 to 90 vol %, preferably 6 to 75 vol %, more preferably 7-50 vol %, even more preferably 8-30 vol %, such as 9-25 vol % CO₂ relative to the total amount of gas present.

Typically, the feed stream will further comprise 10 to 90 vol %, preferably 25-80 vol %, more preferably 35-75 vol % H₂ relative to the total amount of gas present.

In one preferred embodiment, the feed stream comprises 5 to 50 vol % CO₂ relative to the total amount of gas present.

In some embodiments, the H₂ gas is present in the feed stream in an amount of from 10 volume percent (vol %) to 90 vol %, based on combined volume of the H₂ gas and CO₂.

The feed stream may comprise 5 to 90 vol %, preferably 6 to 75 vol %, more preferably 7-50 vol %, even more preferably 8-30 vol %, such as 9-25 vol % CO relative to the total amount of gas present.

In embodiments wherein the feed stream comprises H₂ and CO, the ratio of H₂:CO content in vol % is typically in the range 0.5:1 to 20:1, such as 1:1 to 10:1, wherein the vol % is relative to the total amount of gas present.

In one embodiment, the feed stream may comprise 35-75 vol % H₂ and 9-25 vol % CO₂, wherein each vol % is relative to the total amount of gas present.

In another embodiment, the feed stream may comprise 35-75 vol % H₂ and 9-33 vol % CO, wherein each vol % is relative to the total amount of gas present.

In a particularly preferable embodiment, the feed stream may comprise 35-75 vol % H₂, 2-25 vol % CO₂ and 2-33 vol % CO, wherein each vol % is relative to the total amount of gas present.

Further exemplary compositions for the mixed gaseous feed stream include H₂ contents in the range 65-75 vol %, CO₂ contents in the range 16-23 vol % and CO contents in the range 2-9 vol %.

It will be appreciated that in addition to the gases mentioned above, the gaseous feed stream may comprise further gases. Examples of such further gases include methane, nitrogen, hydrogen sulfide, hydrogen chloride, hydrogen fluoride, sulfur dioxide, carbonyl sulphide, ammonia, oxygen, ethane, ethene and heavy hydrocarbons such as hexane, octane or decane.

The process of the invention comprises contacting the mixed gaseous feed stream as defined herein with a MAPO catalyst as defined herein.

In one embodiment of the process of the invention, the mixed gaseous feed stream is fed into a reaction zone and contacted therein with a MAPO catalyst as disclosed herein. In a particularly preferred embodiment, the mixed gaseous feed stream is contacted with the catalyst in a single reaction zone.

According to some embodiments, an additional stream comprising water, methanol, or other gases, is introduced into the reaction zone with the feed stream. In embodiments, the additional stream may be added to the feed stream prior to introducing the feed stream into the reaction zone, such that a combined stream comprising the feed stream and the additional stream are introduced into the reaction zone simultaneously through the same inlet. In other embodiments, the additional stream may be added to the reaction zone through a different inlet than the feed stream, such that the feed stream and the additional stream are not in contact until both are present in the reaction zone. In either of the above-described embodiments, at some point during the catalyst process both the feed stream and the additional stream are present in the reaction zone and are contacted with the catalyst.

In all embodiments of the invention, it is preferred if the process of the invention takes place in a single reaction zone, i.e. a single reactor.

The temperature in the reaction zone is preferably at least 200° C., more preferably at least 220° C., such as at least 250° C. Typically, the temperature will be less than 450° C., preferably less than 400° C., more preferably less than 375° C., e.g. 325 to 350° C.

The pressure in the reaction zone is preferably at least 5 bar, more preferably at least 10 bar, such as at least 15 bar, e.g. 20 to 30 bar. Typically, the pressure will be less than 50 bar, preferably less than 40 bar.

The MAPO catalysts of the present invention are particularly applicable for heterogeneous catalytic reaction systems (i.e., solid catalyst in contact with a gas reactant). To improve contact surface area, mechanical stability, and/or fluid flow characteristics, the catalysts can be disposed on and/or within a substrate, preferably a porous substrate. In the processes of the present invention, the MAPO catalyst is a solid (i.e. heterogeneous) catalyst.

The processes of the invention involve contacting the mixed gaseous feed stream as defined herein with the MAPO catalyst as defined herein. The catalyst may be used alone, or in combination with one or more additional catalyst materials. Examples of suitable additional catalyst materials are catalysts which are active for the conversion of CO₂ and H₂ to methanol and/or dimethyl ether. Such catalysts are known to those skilled in the art, and may contain elements such as Pt, Pd, Zn, Zr, Ce, In, Cu and O. Typically, these additional catalysts will take the form of a PdZn alloy or a bi- or trimetallic mixed oxide such as Zr₁₋ₓZnₓO₂, Zr_{1-x-y}ZnₓCe_{y}O₂, In—Co, In—Pd, Cu/Zn/ZrO₂ or Cu/ZrO₂. Preferably, the additional catalyst is a bi-metallic mixed oxide comprising Zn and Zr.

Thus, in one embodiment, the process of the invention involves contacting the mixed gaseous feed stream with the catalyst of formula (I) and one or more additional catalysts simultaneously.

In the specific embodiment wherein the process of the invention involves contacting the mixed gaseous feed stream with the catalyst of formula (I) and one or more additional catalysts simultaneously, wherein that additional catalyst is a bi-metallic mixed oxide comprising Zn and Zr, said mixed gaseous feed stream comprises two or more components selected from the group consisting of carbon dioxide, carbon monoxide, hydrogen, methanol and dimethyl ether.

The olefins prepared by the processes of the invention are preferably C2 to C8 olefins, more preferably C2 to C5 olefins, even more preferably C3 to C4 olefins.

The invention will now be described with reference to the following non-limiting examples and figures.

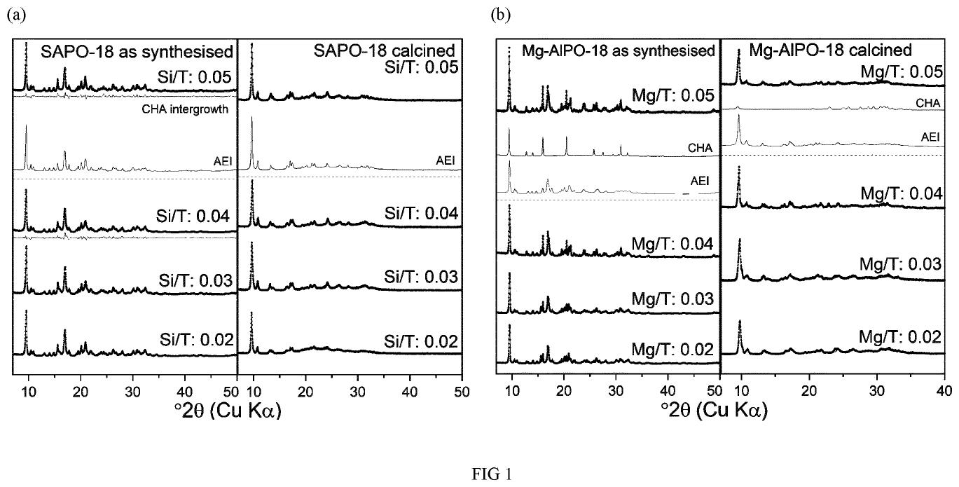

FIG. 1. Powder X-Ray Diffractograms of (a) SAPO-18 and (b) MgAPO-18 catalysts before and after calcination.

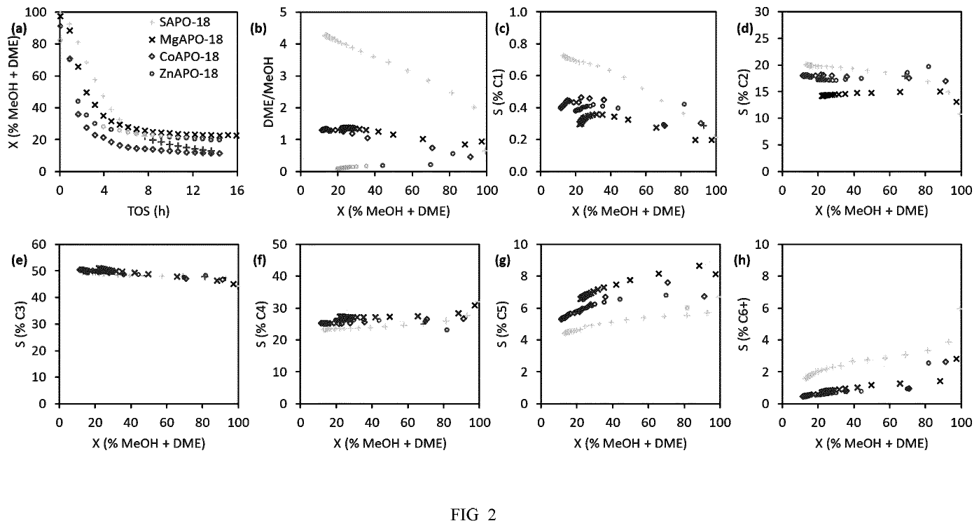

FIG. 2. Catalytic performance of MAPO-18s in MTO reaction conditions. Reaction conditions: 350° C., 1 bar, 0.13 bar MeOH (WHSV=4 g_{MeOH}/g_{cat}/h). (a) Activity in terms % sum of MeOH and DME conversion over time-on-stream. b-h: Product selectivity over conversion. Conversion variation was due to catalyst deactivation during runtime.

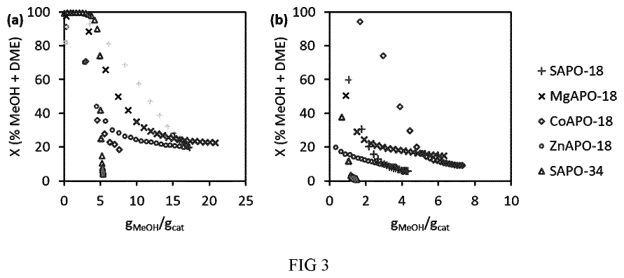

FIG. 3. Catalytic performance of MAPO-18s and commercial SAPO-34 (from ACS Materials). (a) Comparison at 350° C., 1 bar, 0.13 bar MeOH and (b) Comparison at 350° C., 20 bar, 1 bar MeOH.

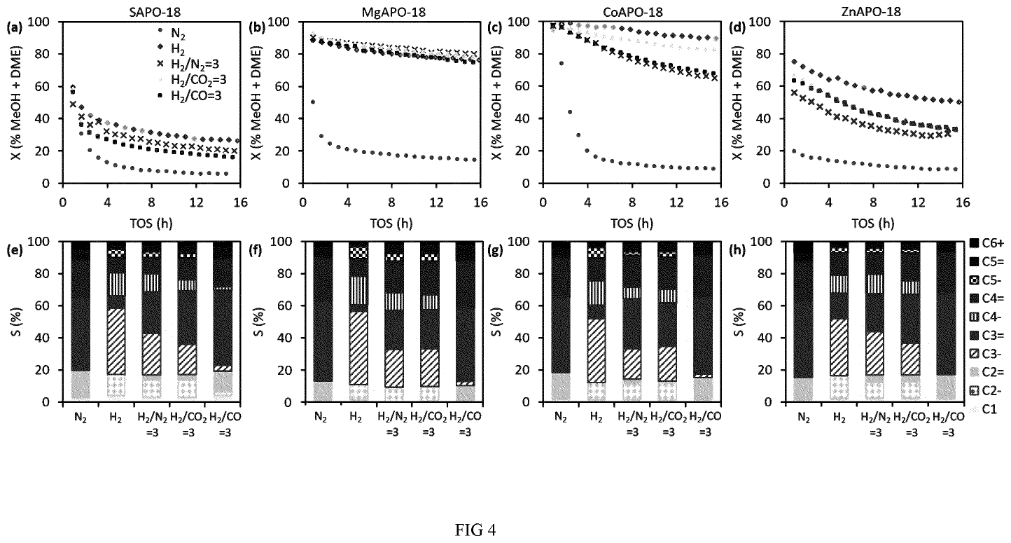

FIG. 4. Catalytic performance of MAPO-18s in various reaction feeds. Reaction conditions: 350° C., 20 bar, 1 bar MeOH (WHSV=2.5 g_{MeOH}/g_{cat}/h), 0.6 bar Ar internal standard, 18.4 bar N₂ or H₂ or H₂/X=3 (in which X=N₂, CO₂ or CO), GHSV≈16 000 mL_{total flow}/mL_{cat.}/h. a-d|Activity in terms % sum of MeOH and DME conversion over time-on-stream for (a) SAPO-18, (b) MgAPO-18, (c) CoAPO-18 and (d) ZnAPO-18. e-h|Product selectivity at 10 h time-on-stream for (e) SiAPO-18, (f) MgAPO-18, (g) CoAPO-18 and (h) ZnAPO-18.

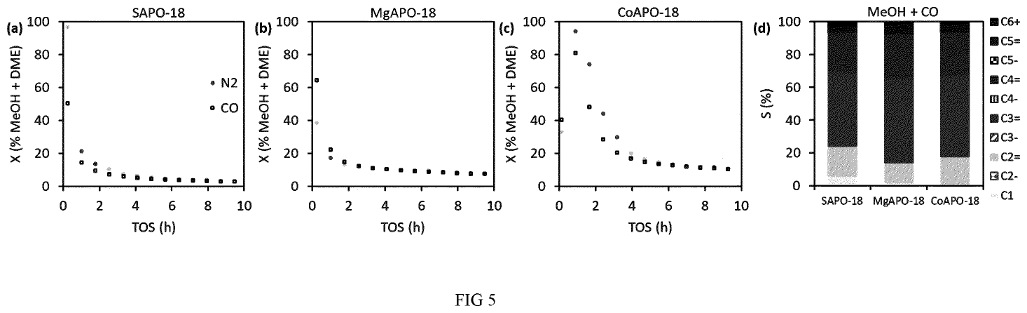

FIG. 5. Catalytic performance of MAPO-18s in MeOH carbonylation reaction. Reaction conditions: 350° C., 20 bar, 1 bar MeOH (WHSV=2.5 g_{MeOH} g_{cat}⁻¹ h⁻¹), 0.6 bar Ar internal standard, 18.4 bar N₂ or CO, GHSV≈16 000 mL_{total flow} mL_{cat}⁻¹ h⁻¹. a-c|Activity in terms % sum of MeOH and DME conversion over time-on-stream for (a) SAPO-18, (b) MgAPO-18, (c) CoAPO-18. (d) Product selectivity at 10 h time-on-stream.

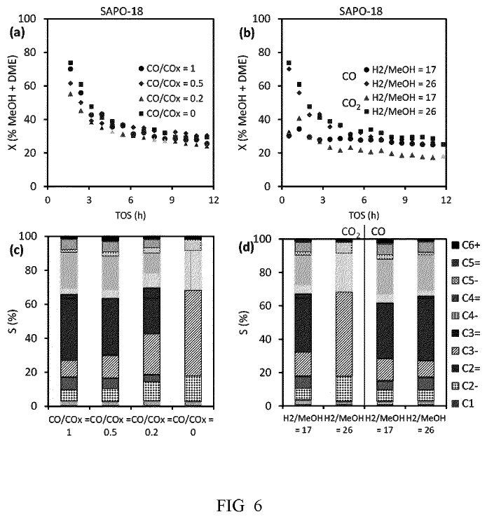

FIG. 6. Effect of CO partial pressure on the catalytic performance of SAPO-18s. Reaction conditions: 350° C., 20 bar, 0.5 bar MeOH (WHSV=2.5 g_{MeOH} g_{cat}⁻¹ h⁻¹), 0.7 bar Ar internal standard, 18.8 bar H₂/COₓ=3, GHSV≈16 000 mL_{total flow} mL_{cat}⁻¹ h⁻¹. (a and b) Activity in terms % sum of MeOH and DME conversion over time-on-stream, and (c and d) Product selectivity at TOS=10 h.

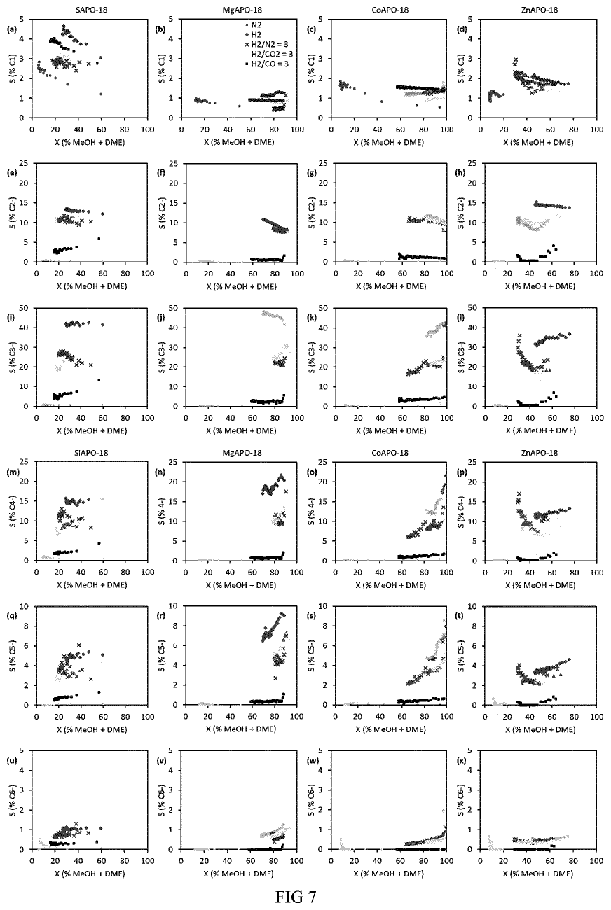

FIG. 7. Paraffin selectivity vs. conversion of MAPO-18s in various reaction feeds. Reaction conditions: 350° C., 20 bar, 1 bar MeOH (WHSV=2.5 g_{MeOH} g_{cat}⁻¹ h⁻¹), 0.6 bar Ar internal standard, 18.4 bar N₂ or H₂ or H₂/X=3 (in which X=N₂, CO₂ or CO), GHSV≈16 000 mL_{total flow} mL_{cat}⁻¹ h⁻¹. Conversion variation was due to catalyst deactivation during runtime.

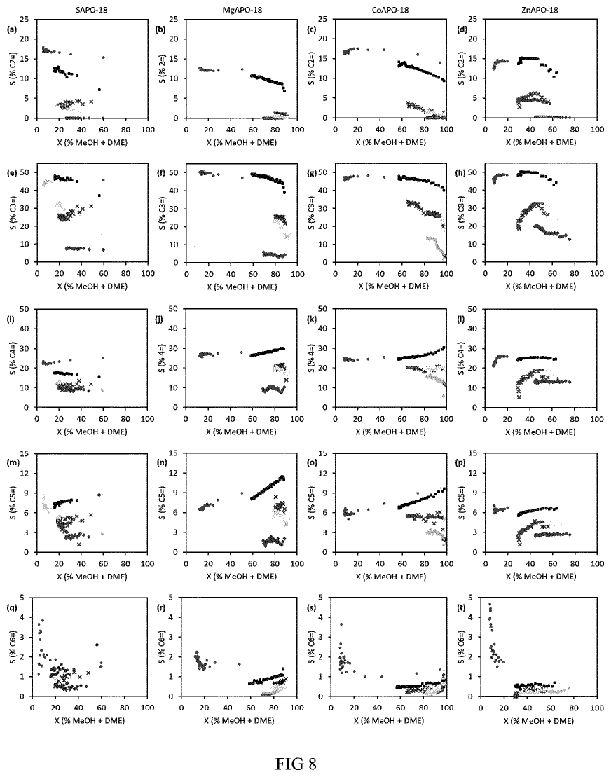

FIG. 8. Olefin selectivity vs. conversion of MAPO-18s in various reaction feeds. Reaction conditions: 350° C., 20 bar, 1 bar MeOH (WHSV=2.5 g_{MeOH} g_{cat}⁻¹ h⁻¹), 0.6 bar Ar internal standard, 18.4 bar N₂ or H₂ or H₂/X=3 (in which X=N₂, CO₂ or CO), GHSV≈16 000 mL_{total flow} mL_{cat}⁻¹ h⁻¹. Conversion variation was due to catalyst deactivation during runtime.

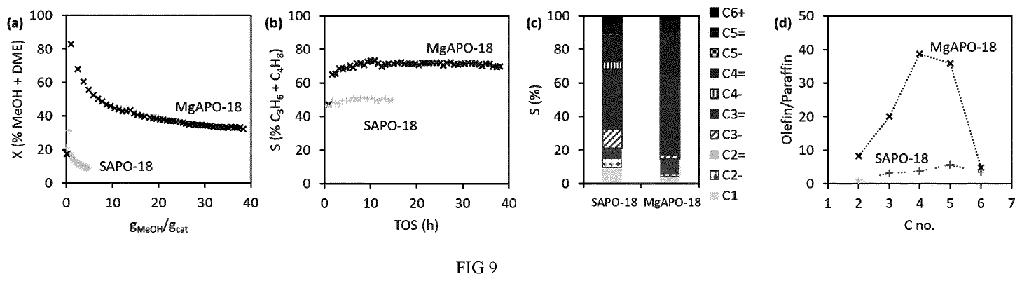

FIG. 9. Catalytic performance of MgAPO-18 and SiAPO-18 in mixed gas feeds at lower temperature of 325° C. Reaction conditions: 325° C., 20 bar, 1 bar MeOH (WHSV=1.7 g_{MeOH}/g_{cat.}/h), 0.6 bar Ar internal standard, 18.4 bar H₂/CO=3, GHSV 10 000 mL_{total flow}/mL_{cat.}/h. (a) Activity over cumulative methanol conversion capacity, (b) sum of propylene and butenes selectivity over time-on-stream, (c) Product selectivity and (d) olefin/paraffin ratio distribution at TOS=10 h.

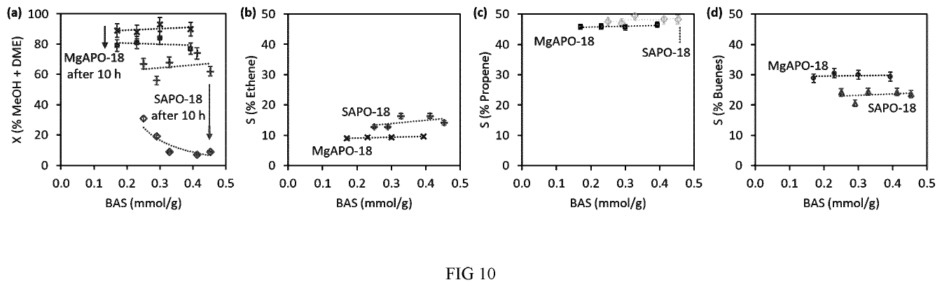

FIG. 10. Catalytic performance of SAPO-18 and MgAPO-18 with varying BAS density. Reaction conditions: 350° C., 20 bar, 1 bar MeOH (WHSV=2.5 g_{MeOH} g_{cat}⁻¹ h⁻¹), 0.6 bar Ar internal standard, 18.4 bar H₂/CO=3, GHSV≈16 000 mL_{total flow} mL_{cat}⁻¹ h⁻¹. (a) Activity in terms % sum of MeOH and DME conversion, (b-d) C₂-C₄ olefins selectivity as a function of BAS density. Symbol size correlates to heteroatom loading (from M/T=0.02 to 0.05) and lines are added to guide the eye.

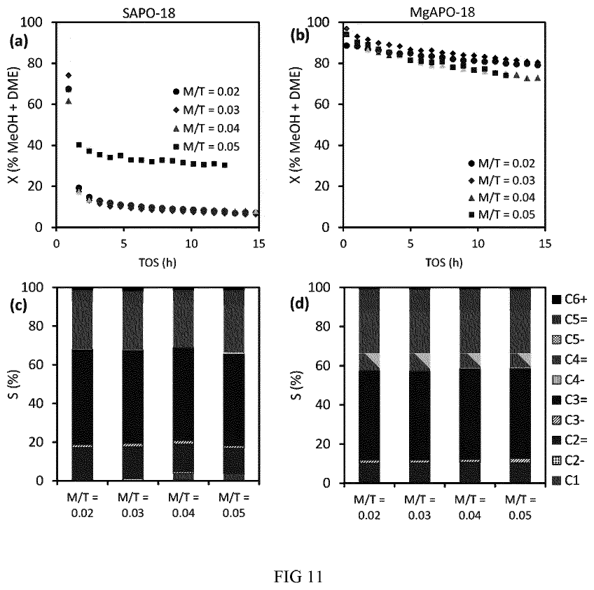

FIG. 11. Catalytic performance of SAPO-18 and MgAPO-18 with varying M/T ratio. Reaction conditions: 350° C., 20 bar, 1 bar MeOH (WHSV=2.5 g_{MeOH} g_{cat}⁻¹ h⁻¹), 0.6 bar Ar internal standard, 18.4 bar H₂/CO=3, GHSV≈16 000 mL_{total flow} mL_{cat}⁻¹ h⁻¹. a-b|Activity in terms % sum of MeOH and DME conversion over time-on-stream for (a) SAPO-18, (b) MgAPO-18. c-d|Product selectivity at TOS=10 h for (c) SAPO-18, (d) MgAPO-18.

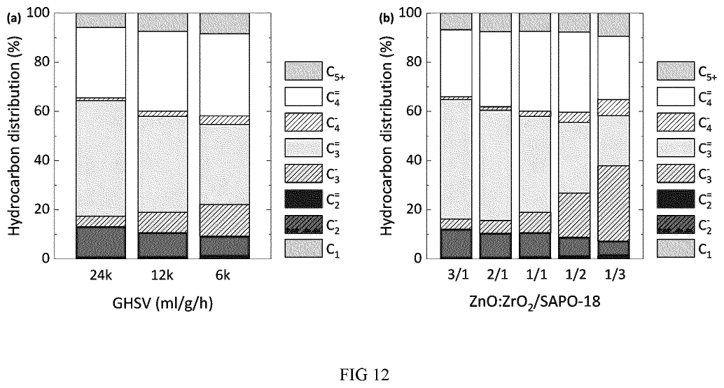

FIG. 12. Hydrocarbon distribution of ZnO:ZrO₂/SAPO-18 catalyst. Reaction conditions: 350° C., 30 bar, H₂/CO₂=3. (a) Varied GHSV with fixed mass loading and mixing of ZnO:ZrO₂/SAPO-18 and (b) Varied mixing of ZnO:ZrO₂/SAPO-18.

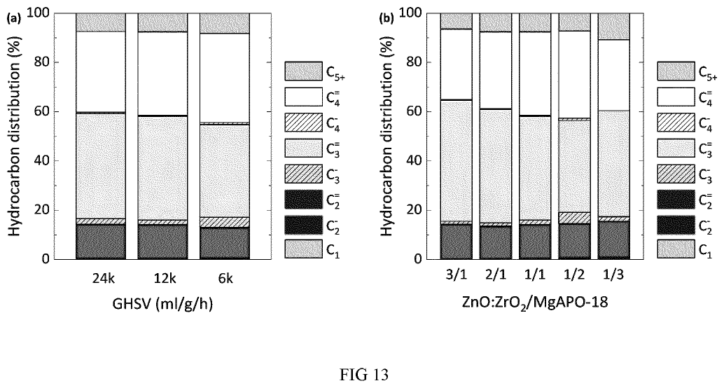

FIG. 13. Hydrocarbon distribution of ZnO:ZrO₂/MgAPO-18 catalyst. Reaction conditions: 350° C., 30 bar, H₂/CO₂=3. (a) Varied GHSV with fixed mass loading and mixing of ZnO:ZrO₂/SAPO-18 and (b) Varied mixing of ZnO:ZrO₂/MgAPO-18.

### EXAMPLES

### Measurement Methods

SEM: The size and morphology of the calcined zeotype particles were analyzed by scanning electron microscopy (SEM), recorded with a Hitachi SU 8230 FE-SEM. The elemental composition was determined utilizing energy-dispersive X-ray spectroscopy (EDS) attached to the same instrument.

N₂-physisorption using BET method: N₂ physisorption was carried out at 77K by using a Belsorp-mini II equipment to determine the BET surface areas and pore volumes. Calcined catalysts were outgassed under vacuum for 4 h, 1 h at 80° C., followed by a period of 3 h at 300° C. The BET surface areas were determined on the basis of a linear fit of the data in the relative pressure (p/po) range of 0.01 to 0.1.

Temperature-programmed desorption of n-propylamine: Temperature-programmed desorption of n-propylamine was performed at atmospheric pressure in a fixed-bed glass reactor (inner diameter 11 mm). Calcined catalysts (250-420 μm) were pretreated at 550° C. under flowing air condition. The catalyst was then cooled to 150° C., after which 80 mL/min N₂ bubbled through a saturator containing n-propylamine at room temperature was then fed to the catalyst for 20 min. The excess amount of n-propylamine was removed by flowing 80 mL/min N₂ for 4 h at 150° C. The temperature was then increased to 550° C. (20° C./min) and the amount of propene desorbed was quantified by using an on-line Pfeiffer Omnistar quadrupole mass spectrometer.

Powder X-ray diffraction: Powder X-ray diffraction patterns of the as-synthesised and calcined MAPOs were measured using a Siemens Bruker D8 Discover instrument with Bragg-Brentano geometry by using Cu K_{α} radiation (X, =1.5406 Å). Samples were mounted on flat sample holders and measured in reflectance mode, Bragg-Brentano geometry. All patterns were fitted using TOPAS6.

### Synthesis of MAPO-18 Catalysts

All catalysts were prepared via hydrothermal synthesis using the same organic structural directing agent, N,N-Diisopropylethylamine (DIPEA, ≥99%, Sigma-Aldrich). The other chemicals were alumina hydrate (A1O(OH), Pural, Sasol), orthophosphoric acid (85% wt. H₃PO₄ in H₂O, Sigma Aldrich), colloidal silica (40% wt. SiO₂ suspension in H₂O, Ludox AS-40, Sigma-Aldrich), magnesium acetate tetrahydrate ((CH₃COO)₂Mg·4H₂O, >98%, Sigma-Aldrich), cobalt(II) acetate tetrahydrate ((CH₃COO)₂Co·4H₂O, >98%, Sigma-Aldrich), zinc acetate dihydrate ((CH₃COO)₂Zn·2H₂O, >98%, Sigma-Aldrich) and deionized water.

SAPO-18 was synthesized with a gel composition of AlO(OH)/SiO₂/H₃PO₄/DIPEA/H₂O=1/0.1/0.9/0.95/9.5. The P source, H₂O and DIPEA were first mixed together. Pural was subsequently added slowly with stirring for 5 minutes, and Ludox was finally added. The synthesis gel was left to stir for 20 minutes to ensure homogeneity. The gel was transferred to a Teflon-lined stainless steel autoclave, and heated at 190° C. under rotation for 12 hours.

MAPO-18 (where M refers to Mg, Co or Zn) were synthesized with identical gel compositions of AlO(OH)/(CH₃COO)₂M/H₃PO₄/DIPEA/H₂O=1/0.1/0.9/0.95/19. The metal acetate precursor was first dissolved in minimal amount of H₂O. The P source, H₂O and DIPEA were then mixed together. Pural was subsequently added slowly with stirring for 5 minutes, and M acetate precursor solution was finally added. The synthesis gel was left to stir for 20 minutes to ensure homogeneity. The gel was transferred to a Teflon-lined stainless steel autoclave (˜50% filled), and heated at 160° C. under rotation for 8 days. All products were washed and centrifuged three times with deionized water and dried at 100° C. for 18 hours. Calcination was performed at 550° C. (3° C./min) under static air condition for 4 h.

Properties of the materials are reported in Table 1 and in FIG. 1.

TABLE 1
Textural and acidic properties of the MAPO-18s.

| | | | | Elemental | | | Density | Brønsted |
|---|---|---|---|---|---|---|---|---|
| | | | | Composition ^{c} | | | of M | Acidity |
| | Crystal | Crystal size | S_{BET} | | | M/ | (mmol/ | (mmol/ |
| | shape ^{a} | (μm) ^{a} | (m²/g) ^{b} | P/Al | M/Al | (Al + P) | g_{cat}) ^{c} | g_{cat}) ^{d} |
| SAPO-18 | rods | ~0.5 | 750 | 1.0 | 0.12 | 0.06 | 0.61 | 0.29 |
| MgAPO-18 | cubes | ~0.5 | 730 | 1.1 | 0.12 | 0.05 | 0.55 | 0.17 |
| CoAPO-18 | cubes | ≤0.5 | 639 | 1.0 | 0.16 | 0.08 | 0.78 | 0.27 |
| ZnAPO-18 | irregular | ~1.0 | 96 | 1.1 | 0.14 | 0.07 | 0.66 | 0.04 |

Properties determined using
^{a} SEM,
^{b} N2-physisorption using BET method,
^{c} SEM-EDS,
^{d} propylamine-TPD.

### Catalytic Performance at MTO Reaction Conditions

The influence of acid strength created by various heteroatom substitution was first evaluated under conditions relevant for the MTO process using an ambient pressure test rig. The reaction conditions were selected to illustrate the ‘true’ deactivation profile of these catalysts, meaning 100% conversion was not sustained over time-on-stream (TOS).

The quantity of calcined catalyst loaded (250-420 μm) was varied depending on the reaction temperature. 100 mg of MAPO-18 was loaded in a fixed-bed U-shaped quartz reactor and heated to 550° C. (5° C./min) in synthetic air feed (N₂/O₂=80/20% v., 25 mL/min). At 550° C., the synthetic air feed was switched to 100% v. 02 feed for 1 h, after which the temperature was decreased to the reaction temperature of 350° C. (2° C./min) in 100% v. N₂ feed. During reaction, methanol was fed to the reactor by passing He through a saturator at 20° C., resulting in a methanol partial pressure of 0.13 bar and WHSV of 4 g_{MeOH}/g_{cat}/h. The total feed flow was 40 mL/min. The effluent from the reactor was analyzed by an online GC-MS instrument (Agilent 7890 with flame ionization detector and 5975C MS detector) equipped with two Restek Rtx-DHA-150 columns. Hydrogen (Praxair, purity 6.0) was used as the carrier gas. Both methanol and dimethyl ether were considered to be reactants when calculating the conversion for activity. Product selectivity was determined based on carbon atoms measured by the FID detector.

From FIG. 2a, SAPO-18 deactivated the slowest in the first 5 h TOS, followed by MgAPO-18, ZnAPO-18 and CoAPO-18. After 5 h TOS, the activity of the MAPO-18s appeared to stabilize at 10-25% conversion.

Referring to FIG. 2b, the DME/MeOH ratio was the highest for SAPO-18, irrespective of conversion level. DME is a product of MeOH dehydration and MeOH and DME are often considered as reactant feed. FIG. 2c to h gives an overview of product selectivity over a range of conversion for all MAPO-18s. All MAPO-18s were selective towards propylene, attaining 49% propylene selectivity. On the other hand, ethylene selectivity varied depending on the heteroatom and was 5% higher for SAPO-18 than MgAPO-18. Correspondingly, methane, butenes and pentenes selectivities were lower for SAPO-18.

FIG. 3a shows the comparison of MAPO-18s with a SAPO-34 purchased from supplier ACS Materials, at MTO reaction conditions of 350° C., 1 bar and MeOH in inert H₂ feed. The M(II)APO-18s performed better than the commercial SAPO-34, but not as efficient as SAPO-18.

### Catalytic Performance at Mixed Feed Reaction Conditions: Effect of Various Reactive Feeds

The influence of various heteroatom substitution was next evaluated under conditions relevant for the mixed feed. Specifically, the reaction pressure was increased from 1 bar to 20 bar and relevant reactants N₂, H₂, CO and CO₂ were co-fed with MeOH.

Methanol conversion over the MAPO-18s at 20 bar in various reactive feeds was investigated using a commercial Microactivity-Effi test rig from PID Eng & Tech. Blank reactor tests were also performed and they showed no reactivity of methanol or CO/CO2. 400 mg of calcined MAPO-18 (250-420 μm) was loaded in a silicon-coated (Silcolloy® coating from SilcoTek) stainless steel reactor with an inner diameter of 6 mm. The catalyst bed (isothermal zone of 5 cm) was supported by glass wool placed above 5 mm glass beads, and a thermocouple (Type K) was inserted in the catalyst bed. The catalyst was heated to the reaction temperature of 350° C. (5° C./min) at 1 bar in 100% v. inert feed (N2 and Ar) for 1 h. The feed flow was then switched to bypass the reactor for 4 h so as to obtain a stable methanol feed flow. N₂ was used to pressurize the methanol liquid feed tank and line, and methanol liquid feed flow was controlled with a Cori Flow controller (Bronkhorst). Methanol was evaporated in the hot box at 140° C. and swept by the flowing gas stream.

Methanol feed flow was 1 g/h, and internal standard Ar feed flow was 7 mLn/min. Individual gas mass flow controllers (Bronkhorst) were used to set the flow rate for each gas, namely CO₂, CO, H₂, N₂, Ar, and the gases were mixed before the methanol feed line. Total feed flow was 220 to 230 mLn/min, resulting in a GHSV of 16 000 h⁻¹. The reaction pressure of 20 bar was controlled by a back pressure regulator after the reactor and this is a PID Eng & Tech patented system based on a high-speed precision servo-controlled valve (VMM01) with eight turns of rotational movement. The product stream was connected to the vent and the online-GC (Scion 456-GC). The GC was equipped with 1 TCD and 2 FID detectors, and 6 columns (MolSieve 13X, HayeSep Q, HayeSep N, Rt-Stabilwax, Rt-Alumina/MAPD and Rtx-1). Helium was used as carrier gas in the TCD channel but N₂ was used as carrier gas in both FID channels.

Results are presented in FIG. 4 and Table 2.

TABLE 2
MeOH conversion capacity and Olefins-to-Paraffins ratios at 350° C., 20
bar, 1 bar MeOH (WHSV = 2.5 g_{MeOH}/g_{cat.}/h), 0.6 bar Ar internal standard,
18.4 bar N₂ or H₂ or H₂/X = 3 (in which X = N₂, CO₂ or CO).

| | (MeOH + DME) conv. capacity | | | | | Olefins/Paraffins | | | | |
|---|---|---|---|---|---|---|---|---|---|---|
| | after 16 h (g_{MeO}H/g_{cat}) | | | | | (C₂₋₄=/−) at 10 h TOS | | | | |
| | | | H2/N₂ = | H₂/CO₂ = | H₂/CO = | | | H₂/N₂ = | H₂/CO₂ = | H₂/CO = |
| | N₂ | H₂ | 3 | 3 | 3 | N₂ | H₂ | 3 | 3 | 3 |
| SAPO-18 | 5.1 | 13.1 | 10.6 | 9.6 | 9.1 | 40.2 | 0.3 | 0.8 | 1.4 | 9.6 |
| MgAPO-18 | 7.6 | 30.2 | 30.2 | 30.3 | 29.1 | 101.0 | 0.2 | 1.1 | 1.1 | 22.8 |
| CoAPO-18 | 8.4 | 34.1 | 28.5 | 32.4 | 28.7 | 81.5 | 0.3 | 1.5 | 1.2 | 22.7 |
| ZnAPO-18 | 4.6 | 22.0 | 13.7 | 17.0 | 16.4 | 95.8 | 0.5 | 0.8 | 1.3 | 91.2 |

The increase in pressure from 1 bar to 20 bar (0.13 vs. 1 bar MeOH in inert N₂ feed) had negligible influence on the deactivation profiles. All MAPO-18s deactivated strongly in the first 5 h TOS and their activities subsequently stabilized at 10-20% conversion. Propylene selectivity over MgAPO-18 was the highest at 49%, followed by CoAPO-18 and ZnAPO-18 at 47%, and SAPO-18 at 44%. In comparison to the other M(II)APO-18s, SAPO-18 showed higher ethylene and C₆₊ selectivities.

Next, H₂ was co-fed with MeOH at high pressures. Remarkably, a comparison between the four materials showed that less H₂ is needed to acquire high, semi-stable activity over MgAPO-18 than over the other three materials (FIG. 4a-d). Furthermore, for SAPO-18, the only M(IV) heteroatom among the four, a steep initial deactivation is observed (FIG. 4a). This suggests that less hydrogen addition may be needed to balance the steady-state HC pool composition in a favourable direction for the non-transition metal catalyst with stronger acid sites, MgAPO-18, followed by Co- and Zn-APO-18.

H₂ does not only hydrogenate coke-precursors, but it also hydrogenates the olefinic products hence decreasing the olefins-to-paraffins ratio (Table 2). Applicable to all MAPO-18 catalysts, the olefins-to-paraffins ratio was lowest for C₂, followed by C₃ and then C₄ (FIG. 4e-h, Table 3).

The simultaneous hydrogenation of olefinic products due to H₂ co-feeding calls for an improvement to this strategy for enlarging lifetime, so we proposed to co-feed CO with H₂ and methanol. Although this approach resulted in a negative effect on the semi-stable conversion level for most materials (FIG. 4a-d), it also led to a dramatic, positive effect on the olefins-to-paraffins ratios for all MAPO-18 catalysts (FIG. 4e-h, Table 3). Strikingly, MgAlPO-18, the non-transition metal material with highest acid strength, showed similar, enhanced conversion level as with H₂ co-feed, yet with olefins-to-paraffins ratios higher than 22 for the C₂-C₄ products (Table 2). From Table 3, the higher DME/MeOH ratios from M(II)APO-18s shows another positive effect of the M(II)APO-18s, which is their ability to catalyse MeOH dehydration under such mixed feed conditions.

Complimentary tests in which CO was co-fed with methanol over the Si-, Mg- and CoAPO-18 catalysts, without H₂ co-feed, showed negligible impact on the activity and product distribution (FIG. 5). The reaction conditions used in these tests mimic the reaction conditions for MeOH carbonylation.

Wider range of reaction conditions, including different CO/COₓ feed ratios and lower methanol partial pressure, were then explored with SAPO-18 (FIG. 6). At those conditions, the ratio between H₂ and hydrocarbon products was higher and almost 100% paraffins selectivity was obtained in the presence of exclusively CO₂ (CO/COₓ=0 in FIG. 6). A clear decrease in paraffins selectivity was observed when CO/COₓ ratio was increased. With lower MeOH partial pressure, i.e. higher H₂/MeOH ratio, paraffin selectivity was also close to 100% in presence of CO₂, yet less than 20% in presence of CO. This affirmed the inhibition of olefin hydrogenation due to the presence of CO, in a wider range of reaction conditions.

FIGS. 7 and 8 are provided to affirm the validity of the above discussion for all conversion levels.

TABLE 3
Catalytic performance of MAPO-18s in various reaction feeds.
Reaction conditions: 350° C., 20 bar, 1 bar MeOH
(WHSV = 2.5 g_{MeOH} g_{cat}⁻¹ h⁻¹), 0.6 bar Ar internal
standard, 18.4 bar N₂ or H₂ or H₂/X = 3 (in which X = N₂,
CO₂ or CO), GHSV ≈16 000 mL_{total flow} mL_{cat}⁻¹ h⁻¹.

| | | Olefin-to- | | | Ethylene/ | DME/ |
|---|---|---|---|---|---|---|
| | Reaction | paraffin ratio | | | Propylene | MeOH |
| Catalyst | Conditions | C2=/− | C3=/− | C4=/− | ratio | ratio |
| | N₂ | 39.2 | 53.6 | 27.2 | 0.38 | 5.4 |
| | H₂ | 0.0 | 0.2 | 0.6 | 0.00 | 4.5 |
| SAPO-18 | H₂/N₂ = 3 | 0.3 | 1.0 | 0.9 | 0.09 | 5.0 |
| | H₂/CO₂ = | 0.4 | 1.8 | 1.9 | 0.12 | 5.0 |
| | 3 | | | | | |
| | H₂/CO = 3 | 5.2 | 13.6 | 8.1 | 0.27 | 4.8 |
| | N₂ | 58.2 | 110.5 | 122.6 | 0.25 | 4.1 |
| | H₂ | 0.0 | 0.1 | 0.6 | 0.00 | 21.6 |
| MgAPO-18 | H₂/N₂ = 3 | 0.1 | 1.0 | 1.8 | 0.04 | 20.1 |
| | H₂/CO₂ = | 0.1 | 1.0 | 2.2 | 0.03 | 21.3 |
| | 3 | | | | | |
| | H₂/CO = 3 | 16.4 | 19.4 | 38.0 | 0.20 | 19.0 |
| | N₂ | 58.1 | 97.3 | 78.6 | 0.36 | 5.1 |
| | H₂2 | 0.0 | 0.2 | 0.9 | 0.00 | — |
| CoAPO-18 | H₂/N₂ = 3 | 0.3 | 1.7 | 2.7 | 0.09 | 21.6 |
| | H₂/CO₂ = | 0.1 | 1.2 | 2.3 | 0.05 | 18.5 |
| | 3 | | | | | |
| | H₂/CO = 3 | 22.8 | 19.9 | 30.6 | 0.28 | 23.9 |
| | N₂ | 57.3 | 115.1 | 101.2 | 0.29 | 0.5 |
| | H₂ | 0.0 | 0.4 | 1.2 | 0.02 | 0.7 |
| ZnAPO-18 | H₂/N₂ = 3 | 0.5 | 0.9 | 1.0 | 0.20 | 0.7 |
| | H₂/CO₂ = | 0.4 | 1.5 | 2.0 | 0.15 | 0.8 |
| | 3 | | | | | |
| | H₂/CO = 3 | 60.8 | 105.8 | 93.6 | 0.30 | 0.8 |

### Catalytic Performance at Lower Temperature

Following the identification of the key descriptors of the catalyst and reaction conditions, we attempted to further close the gap between the optimal conditions of the MTO and methanol synthesis reactions by decreasing the reaction temperature to 325° C. Referring to FIG. 9a, the lower reaction temperature expectedly led to faster deactivation and lower semi-stable conversion levels because the dealkylation of aromatics was unfavourable. Nonetheless, the superior activity and stability of MgAPO-18 over SAPO-18 remained. Importantly, the superior product selectivity of MgAPO-18 over SAPO-18 was convincingly exhibited and maintained at lower temperatures (FIG. 9b-d). From FIG. 9b, selectivity towards propylene and butenes increased during the first 5 h TOS and subsequently stabilized at 72% for MgAPO-18. The selectivity towards propylene and butenes was stable at 50% for SAPO-18, and the 20% difference was due to paraffins (FIG. 9c). This finding was further illustrated in FIG. 9d which shows the olefin-to-paraffin ratio of each hydrocarbon product. Comparing the influence of reaction temperature on the olefin-to-paraffin ratio (FIG. 9d), the olefin-to-paraffin ratios of MgAPO-18 were consistently higher than SAPO-18 at both 350° C. and 325° C. The olefin-to-paraffin ratios of MgAPO-18 appears to be independent of reaction temperature, but those of SAPO-18 clearly decreased at 325° C.

Synthesis Procedure of Additional Catalyst Samples (SAPO-18 and MgAPO-18 with Different Amounts of Si or Mg)

SAPO-18a-d was synthesised by varying Si/T atomic composition in the synthesis gels. The P source, H₂O and DIPEA were first mixed together. Pural was subsequently added slowly with stirring for 5 minutes, and Ludox was finally added. The synthesis gel was left to stir for 20 minutes to ensure homogeneity. The gel was transferred to a Teflon-lined stainless steel autoclave, and heated at 190° C. under rotation for 12 hours.

MgAPO-18a-c were prepared with the same M/T atomic composition in the synthesis gels as SAPO-18a-c. The metal acetate precursor was first dissolved in minimal amount of H₂O. The P source, H₂O and DIPEA were then mixed together. Pural was subsequently added slowly with stirring for 5 minutes, and M acetate precursor solution was finally added. The synthesis gel was left to stir for 20 minutes to ensure homogeneity. The gel was transferred to a Teflon-lined stainless steel autoclave (˜50% filled), and heated at 160° C. under rotation for 8 days. All products were washed and centrifuged three times with deionised water and dried at 100° C. for 18 hours. Calcination was performed at 550° C. (3° C./min) under static air condition for 4 h.

### Characterisation

TABLE 4
Textural and acidic properties of the SAPO-18 and
MgAPO-18 catalysts with varied M/T atomic ratios.
SAPO-18 and MgAPO-18 were catalysts presented
in Table 1 as part of the series of MAPO-18 (M = Si,
Mg, Co and Zn). SAPO-18_a to d and MgAPO-18_a to c were
prepared by varying M/T ratios in the synthesis gel recipes.

| | | | Elemental | | | Density | Brønsted |
|---|---|---|---|---|---|---|---|
| | Crystal | S_{BET} | Composition ^{c} | | | of M | Acidity |
| | size | (m²/ | P/ | M/ | M/T | (mmol/ | (mmol/ |
| | (μm) ^{a} | g) ^{b} | Al | Al | Atoms | g_{cat}) ^{c} | g_{cat}) ^{d} |
| SAPO-18 | ~0.5 | 749 | 1.0 | 0.12 | 0.06 | 0.61 | 0.29 |
| SAPO-18 a | ~0.5 | 729 | 1.0 | 0.06 | 0.03 | 0.51 | 0.33 |
| SAPO-18_b | ~0.5 | 772 | 1.0 | 0.09 | 0.04 | 0.71 | 0.41 |
| SAPO-18_c | ~0.5 | 685 | 0.9 | 0.12 | 0.06 | 0.94 | 0.45 |
| SAPO-18_d | ~0.5 | 749 | 0.9 | 0.12 | 0.06 | 1.01 | 0.25 |
| MgAPO-18 | ~0.5 | 730 | 1.1 | 0.12 | 0.05 | 0.55 | 0.17 |
| MgAPO-18_a | ~0.5 | 748 | 1.0 | 0.06 | 0.03 | 0.49 | 0.23 |
| MgAPO-18_b | ~0.5 | 755 | 1.0 | 0.08 | 0.04 | 0.65 | 0.30 |
| MgAPO-18_c | ~0.5 | 741 | 1.0 | 0.11 | 0.05 | 0.81 | 0.39 |

Properties determined using
^{a} SEM,
^{b} N₂-physisorption using BET method,
^{c} SEM-EDS,
^{d} propylamine-TPD.

### Catalytic Testing

The series of SAPO-18 and MgAPO-18 with varying heteroatom loadings were prepared to elucidate the acidity-performance relations. Results are shown in FIGS. 10 and 11. From FIG. 10a, the higher activity and stability of the stronger acidic MgAPO-18 over SAPO-18 catalysts were upheld over a range of BAS density and heteroatom content. This indicates the stronger influence of acidic strength on performance in comparison to density of acidic sites, thus further supporting the advantage of using M(II) heteroatom substitution. Olefin product selectivities appeared to be independent of BAS density and heteroatom loading (FIG. 10b-d), but with clear distinctions between SAPO-18 and MgAPO-18. Notably, MgAPO-18 enhanced the production of butenes while SAPO-18 produced more ethene. Furthermore, all MgAPO-18 samples maintained high activity after 10 hrs on stream, while the SAPO-18 samples deactivated strongly, hence supporting the conclusions made in earlier sections.

### Mixed Catalyst System

MgAPO-18 and SAPO-18 were mixed with ZnZrO in varying ratios and tested at 350° C., 30 bar, H₂/CO₂=3/1, GHSV=6000-24000 ml/g h. Results obtained after reaching steady-state performance are shown in the Table 5 and in FIGS. 12 and 13. When mixed with ZnZrO, MgAPO-18 and SAPO-18 yielded similar CO₂ conversion and hydrocarbon selectivities under each set of conditions. However, the mixture of ZnZrO with MgAPO-18 yielded higher Propene/Propane product ratios under all sets of conditions, thereby further demonstrating the superior catalytic properties of MgAPO-18 compared to SAPO-18 under mixed feed conditions.

TABLE 5
Results for Mixed catalyst systems. GHSV is
calculated based on the amount of (Catalyst 1 + Catalyst 2).

| | | Cat1: | T | P | GHSV | Conv | C3 prod | | | % C3⁼/ |
|---|---|---|---|---|---|---|---|---|---|---|
| Catalyst 1 | Catalyst 2 | Cat2 | (° C.) | (Bar) | (ml/g/h) | (% CO₂) | (mol/kg/h) | S_{Hydrocarbons} | S_{C3} | C3ₜₒₜ |
| ZnO:ZrO₂ | SAPO-18 | 1:1 | 350 | 30 | 6000 | 9.5 | 0.5 | 54.9 | 25.0 | 71.6 |
| ZnO:ZrO₂ | SAPO-18 | 1:1 | 350 | 30 | 12000 | 7.0 | 0.8 | 57.9 | 27.4 | 82.5 |
| ZnO:ZrO₂ | SAPO-18 | 1:1 | 350 | 30 | 24000 | 4.9 | 1.3 | 60.6 | 31.1 | 91.6 |
| ZnO:ZrO₂ | MgAPO-18 | 1:1 | 350 | 30 | 6000 | 10.1 | 0.5 | 59.9 | 25.0 | 90.1 |
| ZnO:ZrO₂ | MgAPO-18 | 1:1 | 350 | 30 | 12000 | 7.1 | 0.8 | 64.9 | 28.5 | 95.9 |
| ZnO:ZrO₂ | MgAPO-18 | 1:1 | 350 | 30 | 24000 | 5.0 | 1.2 | 63.7 | 28.1 | 94.9 |
| ZnO:ZrO₂ | SAPO-18 | 3:1 | 350 | 30 | 18000 | 7.1 | 1.4 | 60.8 | 32.1 | 92.0 |
| ZnO:ZrO₂ | SAPO-18 | 2:1 | 350 | 30 | 16000 | 7.5 | 1.3 | 64.0 | 32.0 | 89.7 |
| ZnO:ZrO₂ | SAPO-18 | 1:1 | 350 | 30 | 12000 | 7.0 | 0.8 | 57.9 | 27.4 | 82.5 |
| ZnO:ZrO₂ | SAPO-18 | 1:2 | 350 | 30 | 8000 | 6.8 | 0.5 | 55.7 | 26.1 | 61.5 |
| ZnO:ZrO₂ | SAPO-18 | 1:3 | 350 | 30 | 6000 | 7.2 | 0.4 | 52.5 | 26.8 | 39.9 |
| ZnO:ZrO₂ | MgAPO-18 | 3:1 | 350 | 30 | 18000 | 7.8 | 1.3 | 53.2 | 26.7 | 97.6 |
| ZnO:ZrO₂ | MgAPO-18 | 2:1 | 350 | 30 | 16000 | 7.6 | 1.2 | 61.0 | 28.9 | 97.1 |
| ZnO:ZrO₂ | MgAPO-18 | 1:1 | 350 | 30 | 12000 | 7.1 | 0.8 | 64.9 | 28.5 | 95.9 |
| ZnO:ZrO₂ | MgAPO-18 | 1:2 | 350 | 30 | 8000 | 6.9 | 0.5 | 61.1 | 25.6 | 89 |
| ZnO:ZrO₂ | MgAPO-18 | 1:3 | 350 | 30 | 6000 | 6.4 | 0.3 | 59.9 | 24.6 | 93.8 |

1. A process for preparing olefins from a mixed gaseous feed stream, wherein said mixed gaseous feed stream comprises three or more components selected from the group consisting of carbon dioxide, carbon monoxide, hydrogen, methanol and dimethyl ether, said process comprising contacting the mixed gaseous feed stream with a catalyst of formula (I):
M(II)ₓAl₁₋ₓPO₄ (I)
wherein M(II) is a divalent metal ion; and
x=0.002 to 0.5.

2. A process as claimed in claim 1, wherein M(II) is selected from the group consisting of Be, Mg, Ca, Zn, Mn, Fe, Co, Ni, Cu, Cd and Sr, preferably Mg, Co, Zn, Fe, Ni, Cu and Mn, more preferably Mg, Co and Zn, even more preferably Mg.

3. A process as claimed in claim 1 or 2, wherein the catalyst has a pore structure with an eight member ring pore opening.

4. A process as claimed in any of claims 1 to 3, wherein the catalyst has AEI, CHA, AFN or SAV topology

5. A process as claimed in any of claims 1 to 4, wherein said olefins are C2 to C5 olefins, preferably C3 to C4 olefins.

6. A process as claimed in any of claims 1 to 5, wherein said mixed gaseous feed stream is contacted with the catalyst in a single reaction zone.

7. A process as claimed in any of claims 1 to 6, wherein said mixed gaseous feed stream comprises methanol and/or dimethyl ether.

8. A process as claimed in any of claims 1 to 7, wherein said mixed gaseous feed stream comprises carbon dioxide and hydrogen, and optionally carbon monoxide.

9. A process as claimed in any of claims 1 to 7, wherein said mixed gaseous feed stream comprises hydrogen and carbon monoxide.

10. A process as claimed in any of claims 1 to 9, wherein said mixed gaseous feed stream is contacted with the catalyst at a temperature of less than 400° C., preferably less than 375° C.

11. A process as claimed in any of claims 1 to 10, wherein said mixed gaseous feed stream is contacted with the catalyst at a pressure of at least 5 bar, preferably at least 10 bar.

12. A process as claimed in any of claims 1 to 11, wherein said mixed gaseous feed stream is contacted with the catalyst of formula (I) and one or more additional catalysts simultaneously.

13. A process as claimed in claim 12, wherein the one or more additional catalysts is a bi-metallic mixed oxide comprising Zn and Zr.

14. A process for preparing olefins from a mixed gaseous feed stream, wherein said mixed gaseous feed stream comprises two or more components selected from the group consisting of carbon dioxide, carbon monoxide, hydrogen, methanol and dimethyl ether, said process comprising contacting the mixed gaseous feed stream with a catalyst of formula (I):
M(II)xAl1-xPO4 (I)
wherein M(II) is a divalent metal ion; and
x=0.002 to 0.5.
and one or more additional catalysts simultaneously, wherein said one or more additional catalysts is a bi-metallic mixed oxide comprising Zn and Zr

15. A process as claimed in claim 14, wherein said process is as defined in any of claims 2 to 11.

16. The use of a compound of Formula (I) as defined in any of claims 1 to 4 as a catalyst in a process for preparing olefins from a mixed gaseous feed stream as defined in any of claims 1 or 5 to 13.
